(19) United States
(12) Plant Patent Application Publication
(10) Pub. No.: US 2017/0188496 P1
(43) Pub. Date: Jun. 29, 2017

(54) HYDRANGEA PLANT NAMED 'MCKAY'

(50) ;

(71) Applicant: CP DELAWARE, INC., Wilmington, DE (US)

(72) Inventor: Thomas J. BUECHEL, Lake Mills, WI (US)

(73) Assignee: CP DELAWARE, INC., Wilmington, DE (US)

(21) Appl. No.: 14/757,788

(22) Filed: Dec. 29, 2015

(51) Int. Cl. A01H 5/02

(52) U.S. Cl. PLT/250

(57) ABSTRACT

A new and cultivar of Hydrangea macrophylla was created by open pollination followed by selection. A compact rounded growth habit is displayed. Attractive deep rose blossoms are formed in abundance that contrast nicely with textured dark green foliage. Resistance to powdery mildew has been displayed during observations to date. Superior winter hardiness to at least U.S.D.A. Hardiness Zone No. 5 has been experienced.

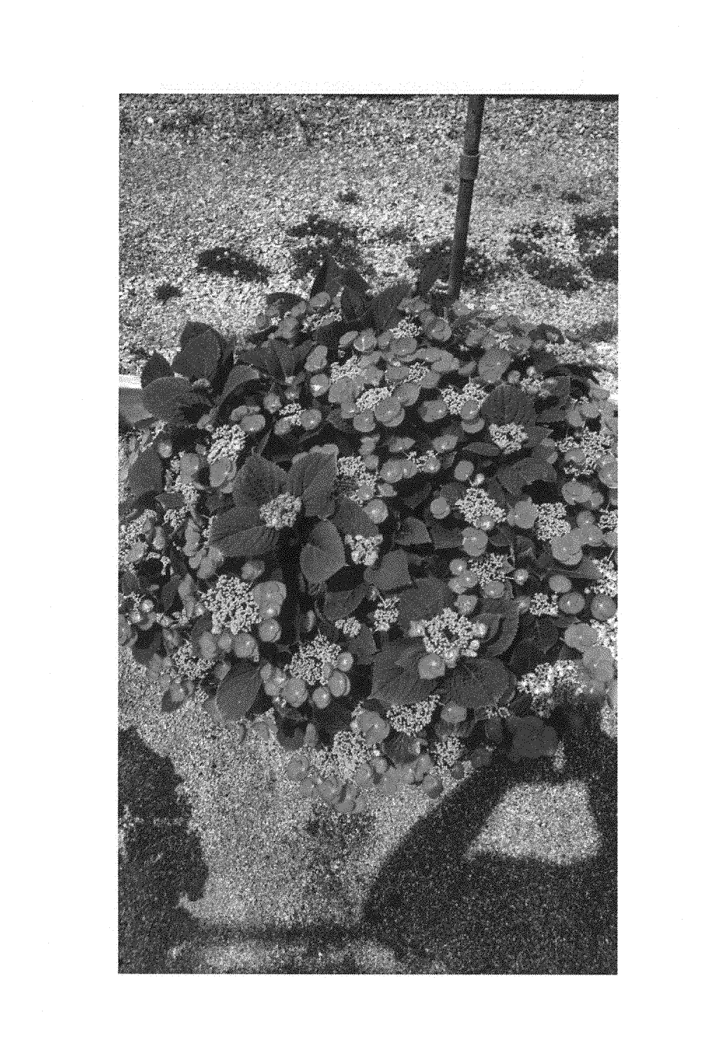

Botanical/commercial classification: Hydrangea macrophylla/Hydrangea Plant. cv. McKay.

### SUMMARY OF THE INVENTION

The new Hydrangea cultivar was developed at Waterloo, Wis., U.S.A. through the open pollination of diverse Hydrangea macrophylla plants followed by selection. The diverse parents were derived from Europe and were unnamed and non-patented. Seeds resulting from such open pollination were sown in containers during 2006. The resulting plants which were physically and biologically different from each other and were grown outdoors in containers under conditions typical of the harsh winter environment of the area. During July 2009 selective study resulted in the identification of a single new plant of the present cultivar. The selection was made on the basis of superior hardiness, excellent vigor, the display of pleasing inflorescence coloration, and the resistance to powdery mildew. Had this new plant not been selected and preserved it would have been lost to mankind.

It was found that the new Hydrangea_plant of the present invention:
(a) forms in abundance attractive deep rose-colored lacecaps inflorescences,
(b) exhibits a compact rounded growth habit,
(c) displays attractive textured dark green foliage,
(d) displays resistance to powdery mildew during observations to date, and
(e) displays superior winter hardiness to at least U.S.D.A. Hardiness Zone No. 5.

The winter hardiness and resistance to powdery mildew are considered to be particularly noteworthy. The deep rose blossoms contrast nicely with the dark green foliage coloration.

The new cultivar well meets the needs of the horticultural industry and can be grown to advantage where attractive ornamentation is to be provided.

The new cultivar has been found to undergo asexual propagation through successive generations by the rooting of softwood cuttings at Waterloo, Wis., U.S.A., and at West Grove, Pa., U.S.A. The new cultivar reproduces well through the use of softwood cuttings. Such asexually propagation has shown that the characteristics of the new cultivar are stable and are strictly transmissible from one generation to another. Accordingly, the new cultivar undergoes asexual propagation in a vine-to-type manner.

The new cultivar has been named ‘McKay’.

### BRIEF DESCRIPITION OF THE PHOTOGRAPH

The accompanying photograph shows as nearly true as it is reasonably possible to make the same in a color illustration of this character, a typical specimen of the new cultivar. A flowering plant of the new cultivar having an age of approximately one age while growing outdoors in a three-gallon container at Waterloo, Wis., U.S.A. is shown from above. The attractive deep rose blossoms and dark green foliage are shown.

The new ‘McKay’ cultivar has not been observed under all possible environmental conditions to date. Accordingly, it is possible that the phenotypic expression may vary somewhat by changes in light intensity and duration, cultural practices, and other environmental conditions.

I claim:

1. A new and distinct Hydrangea plant characterized by the following combination of characteristics:
(a) forms in abundance attractive deep rose-colored lacecap inflorescences,
(b) exhibits a compact rounded growth habit,
(c) displays attractive textured dark green foliage,
(d) displays resistance to powdery mildew during observations to date, and
(e) displays superior winter hardiness to at least U.S.D.A. Hardiness Zone No. 5;
substantially as illustrated and described.
